(19) United States
(12) Patent Application Publication
(10) Pub. No.: US 2003/0143168 A1
(43) Pub. Date: Jul. 31, 2003

(54) Cosmetic compositions comprising at least one PEG fatty acid diester

(76) Inventor: Nathalie Geffroy, Westfield, NJ (US)

Correspondence Address: Finnegan, Henderson, Farabow,, Garrett & Dunner, L.L.P., 1300 I Street, N.W., Washington, DC, 20005-3315, US

(21) Appl. No.: 10/059,155

(22) Filed: Jan. 31, 2002

(51) Int. Cl. A61K 7/021

(52) U.S. Cl. 424/63

(57) ABSTRACT

Compositions comprising at least one PEG fatty acid diester and at least one salt of a fatty acid gelling agent wherein the at least one PEG fatty acid diester and the at least one salt of a fatty acid gelling agent are present in an amount effective to provide a stable composition, and methods for using the same.

The present invention relates to compositions and methods for using the same, such as, for example, methods for care of, for treating and for making-up at least one keratinous material, in particular at least one human keratinous material, such as skin, including the scalp, lips, eyelashes, hair, nails and eyebrows, comprising at least one PEG fatty acid diester and at least one salt of a fatty acid gelling agent wherein the at least one PEG fatty acid diester and the at least one salt of a fatty acid gelling agent are present in an amount effective to provide a stable composition.

Gelling agents may structure compositions and may allow such compositions to be cast, for example, in the form of a stick. Further, structured compositions may, for example, make it possible to control the exudation of components of the composition from the compositions including exudation in a wet or hot atmosphere or environment. However, certain compositions which may be structured or cast in the form of a stick may have certain disadvantages. Disadvantages of such compositions may include, for example, low slip, short play time, drag upon application, very little payoff, lack of uniformity upon application, and a dry afterfeel.

U.S. Pat. No. 5,783,657, for example, describes structuring a composition by using a polyamide in a stick form. However, such a stick composition is usually not mechanically and/or thermally stable. Indeed, a part of the oil contained in such a composition tends to go outside or exude from the stick. Further, when the stick is applied on the skin or lips, the stick may be broken.

Further, cosmetic sticks known in the art may comprise, inter alia, carraghenan, sodium stearate, and PEG 100 stearate. However, such compositions may display at least one of the aforementioned disadvantages.

The inventors have found that the use of at least one specific PEG fatty acid diester wherein the fatty acid comprises a carbon chain of C₂₀ or greater may make it possible to obtain a stable composition. For example, a composition comprising at least one PEG dibehenate (C₂₀) and at least one salt of a fatty acid gelling agent may result in a stable composition. The inventive compositions may further possess at least one property chosen from thermoreversibility, moldability, smooth texture, cooling effect upon application, good payoff, good play time, good coverage, uniform coverage, good slip, and lack of drag upon application. In contrast, a composition solely comprising PEG distearate (C₁₈) may not be both stable and possess at least one of the above desired characteristics.

Note that as used herein, “at least one” means one or more and thus includes individual components as well as mixtures/combinations. Further, as used herein, “keratinous material” is meant to comprise hair, lips, skin, scalp and superficial body growths such as eyelashes, eyebrows and nails.

Further, as used herein, a “salt of a fatty acid gelling agent” refers to a compound that is in the form of a salt at least before it is combined with other components of the inventive composition. Thus, for example, the at least one salt of a fatty acid gelling agent may or may not be in the form of a salt when contained in the inventive composition. In one embodiment, at least a portion of the at least one salt of a fatty acid gelling agent is in the form of a salt when it is present in the inventive composition, such as, for example, 1% of the total amount of the at least one salt of a fatty acid gelling agent by weight is in the form of a salt, such as 10% of the total amount by weight, and further such as 50% of the total amount by weight. In another embodiment, the inventive composition comprises at least one dissociated salt of a fatty acid gelling agent.

As used herein, a “dissociated salt” refers to a salt which has been broken into simpler constituent parts (e.g., cation and anion), for example, by electrolytic dissociation.

Further, as used herein, a “gelling agent” refers to at least one entity chosen from compounds and compositions, wherein the at least one entity, upon mixing with an effective amount of water followed by heating the mixture, is capable of forming a gel upon cooling to room temperature (about 25° C.). As used herein, a “gel” refers to a composition in which at least one disperse phase has combined with the continuous phase, resulting in an increase in the viscosity of the composition. In one embodiment, the gel may be chosen from gels having a viscosity ranging from 1,000 centipoise to 800,000 centipoise at 25° C. as measured using a Brookfield viscometer and gels having a gel strength ranging from 10 g/cm² to 5,000 g/cm² at 25° C. as measured using a TA.XT2i texture analyzer with a ½ inch diameter cylindrical probe.

As defined herein, “stability” is tested by placing the composition standing up in a controlled environment chamber for a specific amount of time at a specific temperature, such as, for example, for 4 weeks at 37° C. As used herein, “standing up” means upright, in a vertical position. For example, if the composition is in the form of a stick, the stick is placed upright in the chamber, i.e., in a vertical position with respect to the surface of the chamber on which the stick is placed.

In this stability test, the physical condition of the sample is inspected as it is placed in the chamber. The sample is then inspected after a specific length of time after the sample is placed in the chamber, such as 24 hours, 1 week, 2 weeks, 3 weeks, 4 weeks, 5 weeks, 6 weeks, 7 weeks, and/or 8 weeks, wherein the temperature of the chamber is set at 4° C., 25° C., 37° C., or 45° C. Each sample may also be tested under freeze-thaw conditions, where the sample is frozen for 12 hours and then allowed to thaw for 12 hours. Generally, freeze-thaw conditions comprise 3 cycles of each of the aforementioned 12 hour periods. At each inspection, the sample is examined for at least one abnormality in the composition such as, for example, bending or leaning if the composition is in stick form, phase separation, melting, or syneresis. As used herein, “syneresis” is the appearance of droplets on a surface of a composition that are visible to the naked eye.

A composition is considered to lack stability if at least one abnormality that impedes functioning of the composition for its intended purpose is observed. The skilled artisan will readily recognize at least one abnormality that impedes functioning of a composition based on the intended application, such as, for example, the appearance of at least one abnormality as described above. The skilled artisan will also readily recognize that the observation of at least one abnormality that impedes functioning of a composition will depend not only on its intended application, but its composition as well. Thus, for example, a composition intended to be a cosmetic stick suitable for commercial application is generally considered to lack stability if at least one abnormality that impedes functioning of the composition as described above is observed after standing up in a controlled environment chamber for 4 weeks at 37° C. Further for example, if such a composition, that is, a composition suitable for commercialization, comprises at least 50% water, such a composition is generally considered to lack stability if at least one abnormality that impedes functioning of the composition as described above is observed after standing up in a controlled environment chamber for 1 week at 37° C.

In one embodiment, the present invention provides a composition comprising: (i) at least one PEG fatty acid diester of formula (1):

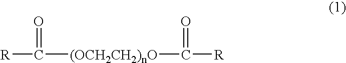

wherein:

R is chosen from (a) alkyl groups comprising at least 19 carbon atoms, wherein the alkyl groups are chosen from linear alkyl groups, branched alkyl groups, and cyclic alkyl groups, and further wherein the alkyl groups are optionally substituted; and (b) alkenyl groups comprising at least 19 carbon atoms, wherein the alkenyl groups are chosen from linear alkenyl groups, branched alkenyl groups, and cyclic alkenyl groups, and further wherein the alkenyl groups are optionally substituted; and

n is 100 or greater; and

(ii) at least one salt of a fatty acid gelling agent,

wherein the at least one PEG fatty acid diester and the at least one salt of a fatty acid gelling agent are present in an amount effective to provide a stable composition.

As used herein, the term “alkyl group” refers to substituted linear alkyl groups, unsubstituted linear alkyl groups, substituted branched alkyl groups, unsubstituted branched alkyl groups, substituted cyclic alkyl groups and unsubstituted cyclic alkyl groups, wherein the alkyl groups comprise at least one carbon and may optionally further comprise at least one heteroatom intercalated in the alkyl chain.

Similarly, as used herein, the term “alkenyl group” refers to substituted linear alkenyl groups, unsubstituted linear alkenyl groups, substituted branched alkenyl groups, unsubstituted branched alkenyl groups, substituted cyclic alkenyl groups and unsubstituted cyclic alkenyl groups, wherein the alkenyl groups comprise at least one carbon and at least one double bond, and may optionally further comprise at least one heteroatom intercalated in the alkenyl chain.

As used herein, “substituted” means comprising at least one substituent. Non-limiting examples of substituents include atoms, such as oxygen atoms and nitrogen atoms, as well as groups, such as hydroxyl groups, ether groups, oxyalkylene groups, polyoxyalkylene groups, carboxylic acid groups, amine groups, amide groups, halogen containing groups, ester groups, siloxane groups, and polysiloxane groups.

In another embodiment, the present invention provides a foundation, a lipstick, a blusher, a make-up-removing product, a make-up product for the body and/or hair, an eyeshadow, an eyeliner, a concealer product, an antisun product or a care product for the skin, lips or hair comprising (i) at least one PEG fatty acid diester of formula (1) wherein R is chosen from alkyl groups comprising at least 19 carbon atoms and alkenyl groups comprising from at least 19 carbon atoms, and n is 100 or greater, and (ii) at least one salt of a fatty acid gelling agent, wherein the at least one PEG fatty acid diester and the at least one salt of a fatty acid gelling agent are present in an amount effective to provide a stable composition.

In another embodiment, the present invention provides a composition comprising (i) at least one PEG fatty acid diester of formula (1) wherein R is chosen from alkyl groups comprising at least 19 carbon atoms and alkenyl groups comprising from at least 19 carbon atoms, and n is 100 or greater, (ii) at least one salt of a fatty acid gelling agent, and (iii) water, wherein the at least one PEG fatty acid diester and the at least one salt of a fatty acid gelling agent are present in an amount effective to provide a stable composition, and further wherein the water is present in an amount greater than 50% by weight relative to the total weight of the composition.

In yet another embodiment, the present invention relates to a method for caring for, making up and/or treating at least one keratinous material comprising applying to the at least one keratinous material a cosmetic composition comprising (i) at least one PEG fatty acid diester of formula (1) wherein R is chosen from alkyl groups comprising at least 19 carbon atoms and alkenyl groups comprising from at least 19 carbon atoms, and n is 100 or greater, and (ii) at least one salt of a fatty acid gelling agent, wherein the at least one PEG fatty acid diester and the at least one salt of a fatty acid gelling agent are present in an amount effective to provide a stable composition.

In another embodiment, the present invention provides a method for modifying the structure of a cosmetic composition which comprises at least one salt of a fatty acid gelling agent, comprising including in the cosmetic composition at least one PEG fatty acid diester of formula (1) wherein R is chosen from alkyl groups comprising at least 19 carbon atoms and alkenyl groups comprising from at least 19 carbon atoms, and n is 100 or greater in an amount effective to provide a stable composition.

In yet another embodiment, the present invention provides a method for providing stability to a cosmetic composition, comprising including in the cosmetic composition (i) at least one PEG fatty acid diester of formula (1) wherein R is chosen from alkyl groups comprising at least 19 carbon atoms and alkenyl groups comprising from at least 19 carbon atoms, and n is 100 or greater and (ii) at least one salt of a fatty acid gelling agent, wherein the at least one PEG fatty acid diester and the at least one salt of a fatty acid gelling agent are present in an amount effective to provide a stable composition.

In yet another embodiment, the present invention provides a method of making a cosmetic composition comprising, adding to the cosmetic composition (i) at least one PEG fatty acid diester of formula (1) wherein R is chosen from alkyl groups comprising at least 19 carbon atoms and alkenyl groups comprising from at least 19 carbon atoms, and n is 100 or greater and (ii) at least one salt of a fatty acid gelling agent, wherein the at least one PEG fatty acid diester and the at least one salt of a fatty acid gelling agent are present in an amount effective to provide a stable composition.

The present invention provides, in another embodiment, a composition comprising (i) at least one PEG fatty acid diester of formula (1) wherein R is chosen from alkyl groups comprising at least 19 carbon atoms and alkenyl groups comprising from at least 19 carbon atoms, and n is 100 or greater and (ii) at least one dissociated salt of a fatty acid gelling agent, wherein the at least one PEG fatty acid diester and the at least one dissociated salt of a fatty acid gelling agent are present in an amount effective to provide a stable composition.

In yet another embodiment, the present invention provides a composition formed from (i) at least one PEG fatty acid diester of formula (1) wherein R is chosen from alkyl groups comprising at least 19 carbon atoms and alkenyl groups comprising from at least 19 carbon atoms, and n is 100 or greater and (ii) at least one salt of a fatty acid gelling agent, wherein the at least one PEG fatty acid diester and the at least one salt of a fatty acid gelling agent are included in the composition in an amount effective to provide a stable composition. As used herein, the phrase “formed from”, is open ended and does not limit the components of the composition to (i) and (ii). Furthermore, the phrase “formed from” does not limit the order of adding components to the composition or require that (i) and (ii) be added to the composition before any other components.

It is to be understood that both the foregoing general description and the following detailed description are exemplary and explanatory only and are not restrictive of the invention, as claimed.

One subject of the invention is cosmetic and/or dermatological compositions which are useful for the care, make-up and/or treating of at least one keratinous material wherein the at least one PEG fatty acid diester and the at least one salt of a fatty acid gelling agent are present in an amount effective to provide a stable composition.

In one embodiment, the inventive compositions are thermoreversible and moldable. Moldable compositions in the form of cosmetic or dermatological products are known in the art. As used herein, a “moldable” composition refers to compositions which are pourable or shapeable when heated but which set or harden as cooled. For example, a moldable composition may be pourable or shapeable when it is heated but, once cooled to room temperature, the composition retains it shape. In one embodiment, a composition is pourable or shapeable when heated to 37° C. or greater.

Moldable compositions may, for example, make it possible to form a product having a desired shape, such, as, for example, a stick form or the form of a container, such as a compact form. Further for example, moldable compositions may make it possible to control the exudation of components that form the compositions, and thus may aid in the storage of such compositions and in the application of cosmetic, dermatological and therapeutic compositions on at least one keratinous material.

However, it is often difficult to produce moldable compositions because, once many compositions are cooled, the shape of the composition is set and subsequent attempts to heat and reshape the compositions result in an inability to mold the composition, i.e., the compositions are not thermoreversible. As used herein, a “thermoreversible” composition refers to a composition that retains the ability to be moldable following at least one heating and cooling cycle. For example, in one embodiment, a composition is thermoreversible if the composition is cooled following mixing or processing and retains its ability to be moldable. As used herein, cooled or cooling cycle refers to any drop in temperature that begins the setting or hardening process.

Thermoreversibility is a characteristic desired by many manufacturers, as thermoreversible compositions may, for example, be more economical and/or more efficient than non-thermoreversible compositions. For example, thermoreversible compositions may be prepared in large batches and then stored in solid form. These compositions may then be reheated at a later time and poured or molded into the desired form or shape. As used herein, a “solid” refers to materials which, when cast in the form of a stick are self-supporting at room temperature (25° C.) and atmospheric pressure (760 mmHg).

Whether a composition is “moldable,” as defined herein, is tested by determining whether a composition, upon heating to 37° C. or greater (such as, for example to a temperature ranging from 50° C. to 75° C.) is pourable or shapeable and whether, upon subsequent cooling, it retains its shape. Whether a composition is “thermoreversible,” as defined herein, is tested by heating and cooling a composition and thereafter determining whether the composition is moldable. A composition is considered to be thermoreversible if, following at least one heating and cooling cycle, it is moldable as defined herein.

The present invention applies not only to make-up products for at least one keratinous material such as lip compositions, lip pencils, foundations including foundations which may be cast in the form of a stick, a dish, a pan or a jar, concealer products, temporary tattoo products, eyeliners, mascara bars, but also to body hygiene products such as deodorant sticks, and to care products and products for treating at least one keratinous material, such as sunscreen and anti-sun products which may be in stick form. The present invention may be in the form of mascara product including mascara bars, an eyeliner product, a foundation product, a lipstick product, a blush for cheeks or eyelids, a deodorant product, a make-up product for the body and/or hair, a make-up-removing product, an eyeshadow product, a face powder product, a concealer product, a treating shampoo product, a hair conditioning product, a sun screen, colorant for the skin or hair, or skin care formula such as, for example, anti-pimple or shaving cut formulas. As defined herein, a deodorant product is a personal hygiene product and does not relate to care, make-up or treatment of keratin materials, including keratin fibers. In one embodiment, the invention composition is not a deodorant product.

In another embodiment, the present invention provides a composition comprising (i) at least one PEG fatty acid diester of formula (1) wherein R is chosen from alkyl groups comprising at least 19 carbon atoms and alkenyl groups comprising from at least 19 carbon atoms, and n is 100 or greater, (ii) at least one salt of a fatty acid gelling agent, and (iii) water, wherein the at least one PEG fatty acid diester and the at least one salt of a fatty acid gelling agent are present in an amount effective to provide a stable composition, and further wherein the water is present in an amount greater than 50% by weight relative to the total weight of the composition. Thus, the compositions of the present invention may impart a cooling sensation to the at least one keratinous material to which it is applied.

In another embodiment, the compositions of the present invention further comprise water in an amount greater than 5% by relative to the total weight of the composition, such as, for example, 30% by weight, 50% by weight, 60% by weight, and further such as 80% by weight.

As previously mentioned, the present invention also relates to a method for modifying the structure of a cosmetic composition which comprises at least one salt of a fatty acid gelling agent, comprising including in the cosmetic composition at least one PEG fatty acid diester of formula (1) wherein R is chosen from alkyl groups comprising at least 19 carbon atoms and alkenyl groups comprising from at least 19 carbon atoms, and n is 100 or greater in an amount effective to provide a stable composition. As used herein, the “structure” of a composition refers to the plasticity of the composition. Further, as used herein, “plasticity” refers to a rheological property of solid or semi-solid materials expressed as the degree to which they will flow or deform under applied stress and retain the shape so induced, either permanently or for a definite time interval. Thus, for example, the inclusion of at least one PEG fatty acid diester of formula (1) wherein R is chosen from alkyl groups comprising at least 19 carbon atoms and alkenyl groups comprising from at least 19 carbon atoms, and n is 100 or greater in a composition which comprises at least one salt of a fatty acid gelling agent, such as, for example, sodium stearate, may plasticize the composition.

In yet another embodiment, the present invention provides a method for providing stability to a cosmetic composition, comprising including in the cosmetic composition (i) at least one PEG fatty acid diester of formula (1) wherein R is chosen from alkyl groups comprising at least 19 carbon atoms and alkenyl groups comprising from at least 19 carbon atoms, and n is 100 or greater and (ii) at least one salt of a fatty acid gelling agent, wherein the at least one PEG fatty acid diester and the at least one salt of a fatty acid gelling agent are present in an amount effective to provide a stable composition.

The compositions of the present invention comprise at least one PEG fatty acid diester of formula (1):

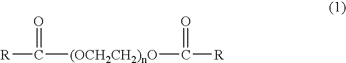

wherein:

R is chosen from (a) alkyl groups comprising at least 19 carbon atoms, wherein the alkyl groups are chosen from linear alkyl groups, branched alkyl groups, and cyclic alkyl groups, and further wherein the alkyl groups are optionally substituted; and (b) alkenyl groups comprising at least 19 carbon atoms, wherein the alkenyl groups are chosen from linear alkenyl groups, branched alkenyl groups, and cyclic alkenyl groups, and further wherein the alkenyl groups are optionally substituted; and

n is 100 or greater.

In another embodiment, R is chosen from alkyl groups comprising from 19 to 31 carbon atoms and alkenyl groups comprising from 19 to 31 carbon atoms. In another embodiment, R is chosen from alkyl groups comprising 19 carbon atoms and alkenyl groups comprising 19 carbon atoms. In another embodiment, R is chosen from alkyl groups comprising 20 carbon atoms and alkenyl groups comprising 20 carbon atoms. In another embodiment, R is chosen from alkyl groups comprising 21 carbon atoms and alkenyl groups comprising 21 carbon atoms, and in another embodiment, R is chosen from alkyl groups comprising 23 carbon atoms and alkenyl groups comprising 23 carbon atoms. In one embodiment, n ranges from 100 to 500. In another embodiment, n is 100. In another embodiment, n is 150. In yet another embodiment, n is 200, while in another embodiment, n is 250, and in yet another embodiment, n is 300. In another embodiment, R is chosen from alkyl groups comprising 21 carbon atoms and alkenyl groups comprising 21 carbon atoms and n is 100. In another embodiment, R is chosen from alkyl groups comprising 21 carbon atoms and alkenyl groups comprising 21 carbon atoms and n is 150.

In one embodiment, the at least one PEG fatty acid diester is present in an amount ranging from 0.001% to 2% by weight relative to the total weight of the composition, such as, for example, ranging from 0.5% to 2% by weight. In another embodiment, the at least one PEG fatty acid diester is present in an amount ranging up to 15% by weight relative to the total weight of the composition. In another embodiment, the at least one PEG fatty acid diester has a melting point ranging from 50° C. to 60° C. In yet another embodiment, the at least one PEG fatty acid diester has a melting point ranging from 52° C. to 57° C.

A non-limiting example of at least one PEG fatty acid diester which may be used in the composition according to the present invention is the commercial product sold by Ethox Chemical under the name ETHOX PEG-6000 DB (I.N.C.I. name: PEG-150 Dibehenate). ETHOX PEG-6000 DB is composed of PEG-150 fatty acid diesters, wherein at least 88% of the fatty acid carbon chains are C₂₀ carbon chains and C₂₂ carbon chains. This product is sold in the form of a solid having a melting point ranging from 52° C. to 57° C.

In one embodiment, the inventive composition comprises at least two PEG fatty acid diesters of formula (1), wherein R is chosen from alkyl groups comprising at least 19 carbon atoms and alkenyl groups comprising at least 19 carbon atoms, and n is 100 or greater.

The compositions of the present invention also comprise at least one salt of a fatty acid gelling agent. Salts of a fatty acid gelling agent suitable for use in the present invention as the at least one salt of a fatty acid gelling agent may be chosen from salts of C₁₀ to C₄₀ fatty acid gelling agents, such as salts of C₁₂ to C₂₂ fatty acid gelling agents, such as salts of C₁₄ to C₂₀ fatty acid gelling agents, and further such as salts of C₁₆ to C₂₀ fatty acid gelling agents. Non-limiting examples of the at least one salt of a fatty acid gelling agent include salts of lauric acid (laurates —C₁₂), salts of myristic acid (myristates-C₁₄), salts of palmitic acid (palmitates-C₁₆), salts of stearic acid (stearates-C₁₈), salts of arachidic acid (arachidates-C₂₀), and salts of behenic acid (behenates-C₂₂). In one embodiment, the at least one salt of a fatty acid gelling agent is chosen from salts of lauric acid, salts of palmitic acid, salts of stearic acid, and salts of behenic acid.

The at least one salt of a fatty acid gelling agent may be chosen from inorganic salts of a fatty acid gelling agent and organic salts of a fatty acid gelling agent. In one embodiment, the at least one salt of a fatty acid gelling agent is chosen from alkali metal salts of a fatty acid gelling agent. In another embodiment, the at least one salt of a fatty acid gelling agent is chosen from sodium salts of a fatty acid gelling agent, such as, for example, sodium laurate, sodium palmitate, sodium stearate, and sodium behenate. In another embodiment, the at least one salt of a fatty acid gelling agent is chosen from potassium salts of a fatty acid gelling agent, such as, for example, potassium laurate, potassium palmitate, potassium stearate, and potassium behenate.

In another embodiment, the at least one salt of a fatty acid gelling agent may be chosen from those derived from reaction of a fatty acid with an amine and those derived from reaction of a fatty acid with alkanolamines. In another embodiment, the at least one salt of a fatty acid gelling agent may be formed in situ, such as, for example, by reaction of a fatty acid with a base.

In one embodiment, the inventive compositions comprise at least two salts of a fatty acid gelling agent. In another embodiment, the at least two salts of a fatty acid gelling agent are chosen from salts of stearic acid and salts of lauric acid.

In another embodiment, the at least one salt of a fatty acid gelling agent is present in an amount ranging from 1% to 30% by weight relative to the total weight of the composition. In another embodiment, the at least one salt of a fatty acid gelling agent is present in an amount ranging from 3% to 15% by weight relative to the total weight of the composition.

The compositions of the present invention may further comprise at least one PEG fatty acid diester of formula (1) wherein R is chosen from alkyl groups comprising less than 19 carbon atoms and alkenyl groups comprising less than 19 carbon atoms, such as from 13 to 18, and/or wherein n is less than 100. For example, in one embodiment, the compositions of the present invention further comprise at least one PEG fatty acid diester of formula (1) wherein R is chosen from alkyl groups comprising from 13 to 18 carbon atoms and alkenyl groups comprising from 13 to 18 carbon atoms. In another embodiment, the inventive compositions further comprise at least one PEG fatty acid diester of formula (1) wherein R is chosen from alkyl groups comprising 17 carbon atoms and alkenyl groups comprising 17 carbon atoms. In yet another embodiment, the inventive compositions further comprise at least one PEG fatty acid diester of formula (1) wherein R is chosen from alkyl groups comprising 18 carbon atoms and alkenyl groups comprising 18 carbon atoms.

Further, in one embodiment, the composition of the present invention may be in a form chosen from a paste, a solid, a gel, and a cream. It may be an emulsion (i.e., an oil-in-water or water-in-oil emulsion), a multiple emulsion (i.e., an oil-in-water-in-oil emulsion or water-in-oil-in-water emulsion), or a solid, rigid or supple gel. The composition of the invention may, for example, comprise an external or continuous fatty phase. Further for example, in another embodiment, the at least one PEG fatty acid diester and the at least one salt of a fatty acid gelling agent are present in a system chosen from an aqueous system, an alcohol aqueous system (including an glycolic aqueous system), a hydro-alcoholic system (including a hydro-glycolic system), an oil-in-water emulsion, a water-in-oil, an oil-in-water-in-oil emulsion, and a water-in-oil-in-water emulsion. As used herein, the term “alcohol” comprises monoalcohols and polyalcohols.

Further, the inventive composition may, for example, be a molded composition or cast as a stick or a dish. The inventive composition, in one embodiment, is a solid, such as a molded stick or a poured stick. In another embodiment, the inventive composition is in a form chosen from molded sticks, poured sticks and gels.

The concentrations of the at least one PEG fatty acid diester and the at least one salt of a fatty acid gelling agent may be chosen according to the desired hardness and desired stability of the compositions and according to the specific application envisaged. The respective concentrations of the at least one PEG fatty acid diester and of the at least one salt of a fatty acid gelling agent can be such that a disintegrable solid which does not flow under its own weight at 25° C. is obtained.

Depending on the intended application, such as a stick, hardness of the composition may also be considered. In one embodiment, the at least one PEG fatty acid diester and the at least one salt of a fatty acid gelling agent are present in an amount effective to further provide hardness to the composition. The hardness of a composition may, for example, be expressed in grams (g). The composition of the present invention may, for example, have a hardness ranging from 10 g to 5000 g, such as from 15 g to 500 g, further such as from 20 g to 600 g, and further such as from 30 g to 150 g.

This hardness is measured as follows. A first test for hardness is according to a method of penetrating a probe into the composition and in particular using a texture analyzer (for example TA-XT2i from Stable Microsystems) equipped with a stainless steel cylinder of height 35 mm and diameter 4 mm. The hardness measurement is carried out at 20° C. at the center of 5 samples of the composition.

The cylinder penetrates at a speed of 2 mm/s, the total displacement being 5 mm.

The recorded hardness value is that of the maximum peak observed. The measurement error is ±10 g.

The hardness of the composition of the present invention may be such that the compositions are self-supporting and can easily disintegrate to form a satisfactory deposit on a keratinous material. In addition, this hardness may impart good impact strength to the inventive compositions which may be molded or cast, for example, in stick or dish form.

The skilled artisan may choose to evaluate a composition using the tests for hardness outlined above based on the application envisaged and the hardness desired.

According to the present invention, the compositions in stick form may also possess the properties of deformable, flexible elastic solids and may also have noteworthy elastic softness upon application to a keratinous material. Further, in one embodiment, the inventive composition has a melting point ranging from 40° C. to 150° C.

The composition of the present invention may also further comprise at least one suitable additive commonly used in the field concerned chosen from fatty materials, waxes, coloring agents, fillers, humectants, texture modifiers, moisturizers, viscosity modifiers, antioxidants, essential oils, preserving agents, fragrances, neutralizing agents, liposoluble polymers, polysaccharides, silicones, fluorinated compounds, and cosmetically active agents and dermatological active agents such as, for example, emollients, vitamins, essential fatty acids and UV-screening agents. In one embodiment, the inventive compositions do not comprise at least one polysaccharide. In another embodiment, the composition of the present invention is transparent and/or clear, including, for example, a composition without pigments. In yet another embodiment, the composition of the present invention is neither transparent nor clear.

Needless to say, the person skilled in the art will take care to select the optional additional additives and the amount thereof such that at least one advantageous property of the composition according to the invention, such as stability, non-migration, is not, or is not substantially, adversely affected by the addition(s) envisaged.

In one embodiment, the at least one suitable additive is chosen from glycerin, glycols such as propylene glycol and butylene glycol, and monosaccharides such as mannitol and sorbitol.

In another embodiment, the compositions of the invention may further comprise at least one fatty material. The at least one fatty material may, for example, be chosen from lipids, gums, resins, polyamides, fatty compounds chosen from compounds which are pasty at room temperature, fatty compounds which are liquid at room temperature, and fatty compounds which are solid at room temperature. Non-limiting examples of the at least one fatty material include lanolins, lanolin derivatives (such as acetylated lanolins, oxypropylenated lanolins, and isopropyl lanolate), esters of fatty acids and esters of fatty alcohols (such as those comprising from 20 to 65 carbon atoms, and such as triisostearyl citrate, cetyl citrate, arachidyl propionate, polyvinyl laurate), cholesterol esters (such as triglycerides of plant origin, such as hydrogenated plant oils (hydrogenated castor oil)), viscous polyesters (such as poly(12-hydroxystearic acid)), and polydimethylsiloxanes (PDMS) having alkyl and/or alkoxy pendant chains comprising from 8 to 24 carbon atoms, such as stearyldimethicones (such as DC2503 and DC25514 (a methyl stearyldimethicone) from Dow Corning)). The at least one fatty material may be present in an amount ranging up to 60% by weight relative to the total weight of the composition, for example from 0.1% to 45% by weight, and, as a further example, from 2% to 30% by weight, in the composition, if present.

According to another embodiment, the compositions of the invention may further comprise at least one wax. At least one wax, for example, may be used to form a non-transparent composition. As used herein, a “wax” may be any lipophilic fatty compound which is soluble in a fatty phase, unlike most fillers or pigments. The at least one wax, for example, may have a melting point greater than about 35° C., such as, for example greater than about 55° C. Non-limiting examples of such waxes include waxes of natural origin, such as beeswax, carnauba wax, candelilla wax, jojoba wax, ouricury wax, Japan wax, cork fiber wax, sugar cane wax, paraffin waxes, lignite wax, microcrystalline waxes, lanolin wax, montan wax and ozokerites, hydrogenated oils such as hydrogenated jojoba oil, jojoba esters, waxes of synthetic origin, such as polyethylene waxes derived from polymerization of ethylene, waxes obtained by Fischer-Tropsch synthesis, fatty acid esters other than the at least one PEG fatty acid diester of formula (1), fatty acid glycerides, and silicone waxes such as derivatives of poly(di)methylsiloxane. In one embodiment, the at least one wax may be present in the composition up to 3%, and in another embodiment at least 3%, such as up to 30% or up to 50%, if present.

Further, as previously mentioned, the compositions of the invention may further comprise at least one coloring agent. The at least one coloring agent may be chosen from pigments, dyes, nacreous pigments, and pearling agents. The at least one coloring agent may be chosen, for example, in order to obtain make-up compositions which give good coverage, that is, which do not leave a significant amount of the at least one keratin material to which it is applied showing through. The at least one coloring agent may also reduce the sticky feel of the compositions, unlike soluble dyes.

Representative liposoluble dyes which may be used according to the present invention include Sudan red, DC Red 17, DC Green 6, β-carotene, soybean oil, Sudan brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%.

The pigments which may be used according to the present invention may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate and ferric blue. Representative examples of organic pigments include carbon black, pigments of D & C type, and lakes based on cochineal carmine, barium, strontium, calcium and aluminum. If present, the pigments may have a concentration ranging up to 40% by weight of the total weight of the composition, such as from 1% to 35%, and further such as from 2% to 25%. In the case of certain products, the pigments, including nacreous pigments, may, for example, represent up to 90% by weight of the composition.

The nacreous pigments (or nacreous pigments) which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, may have a concentration ranging up to 90% by weight of the total weight of the composition, such as from 0.1% to 20%.

Accordingly to the present invention, the inventive compositions may further comprise at least one filler. As used herein, the term “filler” means any particle that is solid at room temperature and atmospheric pressure, used alone or in combination, which does not react chemically with the various ingredients of the composition and which is insoluble in these ingredients, even when these ingredients are raised to a temperature above room temperature and in particular to their softening point or their melting point. In one embodiment, the at least one filler has a melting point at least greater than 170° C., for example, greater than 200° C. In one embodiment, the at least one filler may have an apparent diameter ranging from 0.01 μm to 150 μm, such as from 0.5 μm to 120 μm, for example from 1 μm to 80 μm. An apparent diameter corresponds to the diameter of the circle into which the elementary particle fits along its shortest dimension (thickness for leaflets).

Further, the at least one filler may be absorbent, i.e., capable in particular of absorbing the oils of the composition and also the biological substances secreted by the skin, may be surface-treated, e.g., to make it lipophilic, and/or may be porous so as to absorb the sweat and/or sebum secreted by the skin.

The at least one filler may be chosen from inorganic and organic fillers, and may have any shape such as lamellar, spherical and/or oblong. Non-limiting examples of the at least one inert filler include talc, mica, silica, kaolin, polyamide powders (such as Nylon® powder, and such as the product sold by Atochem as Orgasol®), poly-β-alanine powders, polyethylene powders, acrylic polymer powders (such as polymethyl methacrylate (PMMA) powder, for instance the product sold by Wacker as Covabead LH-85 (particle size 10-12 μm) and the acrylic acid copolymer powder sold by Dow Corning as Polytrap®), polytetrafluoroethylene (Teflon®) powders, lauroyllysine, boron nitride, silica, kaolin, starch, starch derivatives, hollow polymer microspheres (such as those hollow polymer microspheres formed from polyvinylidene chloride and acrylonitrile, for instance the product sold by Nobel Industrie as Expancel®), and polymerized silicone microspheres (such as those polymerized silicone microspheres sold by Toshiba as Tospearl®), precipitated calcium carbonate, magnesium carbonate and hydrocarbonate, hydroxyapatite, hollow silica microspheres (such as the product sold by Maprecos as Silica Beads®), glass microcapsules, ceramic microcapsules, and polyester particles.

The at least one filler may be present in the inventive composition in an amount ranging from 0.1% to 40% relative to the weight of the total composition, such as from 2% to 30%, and, for example, from 5% to 20%, if present.

The compositions according to the present invention may be manufactured by one of ordinary skill in the art. For example, they may be manufactured by a process which comprises heating the at least one PEG fatty acid diester of formula (1) at least to its softening point, adding the at least one salt of a fatty acid gelling agent, in one embodiment in a melted form, and any suitable additives, if present, to the PEG fatty acid diester of formula (1) followed by mixing the composition. Further for example, they may be manufactured by a process which comprises solubilizing the at least one PEG fatty acid diester of formula (1), the at least one salt of a fatty acid gelling agent, and any suitable additives, if present, and mixing the aqueous and solvent phases. The resultant homogeneous mixture may then be cast or poured in a suitable mold such as a lipstick mold, foundation mold, or deodorant mold or cast directly into the packaging articles such as a case, a dish, a pan or a jar.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and in the attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result. The percentages are given on a weight basis.

### Examples

### Example 1

The following composition was prepared.

TABLE 1

| Components | Weight Percent |
|---|---|
| A | |
| Glycerin | 12.00 |
| Propylene glycol | 3.00 |
| Preservatives | 0.75 |
| B | |
| PEG 150 Dibehenate | 1.75 |
| (ETHOX P-6000 DB) | |
| C | |
| Sodium Stearate | 7.00 |
| pH adjuster | 0.35 |
| Nacreous pigments | 14.00 |
| Deionized Water | q.s. to 100 |

The components in phase A were combined with water and mixed.

PEG 150 Dibehenate was then added with stirring and the mixture was heated to 70° C. Once all of the components of the mixture were in solution, the components of phase C were added with stirring. The resultant mixture was then poured in a mold and allowed to cool. A thermoreversible, moldable and stable composition which exhibited a smooth surface, good texture, smooth glide upon application, good coverage, and uniform coverage was obtained.

### Example 2

The following composition was prepared:

TABLE 2

| Component | Weight Percent |
|---|---|
| A | |
| Glycerin | 12.00 |
| Propylene glycol | 3.00 |
| Preservatives | 0.75 |
| B | |
| PEG 150 Dibehenate | 1.75 |
| (ETHOX P-6000 DB) | |
| C | |
| Sodium Stearate | 7.00 |
| Wax | 5.00 |
| pH adjuster | 0.35 |
| Nacreous pigments | 14.00 |
| Deionized Water | q.s. to 100 |

The components in phase A were combined with water and mixed.

PEG 150 Dibehenate was then added with stirring and the mixture was heated to 70° C. Once all of the components of the mixture were in solution, the components of phase C were added with stirring, and the mixture was heated to a temperature sufficient to melt the wax. The resultant mixture was then poured in a mold and allowed to cool. A thermoreversible, moldable and stable composition which exhibited a smooth surface, good texture, smooth glide upon application, good coverage, and uniform coverage was obtained.

What is claimed is:

1. A composition comprising
(i) at least one PEG fatty acid diester of formula (1): 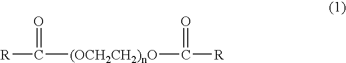
wherein:
R is chosen from (a) alkyl groups comprising at least 19 carbon atoms, wherein said alkyl groups are chosen from linear alkyl groups, branched alkyl groups, and cyclic alkyl groups, and further wherein said alkyl groups are optionally substituted, and (b) alkenyl groups comprising at least 19 carbon atoms, wherein said alkenyl groups are chosen from linear alkenyl groups, branched alkenyl groups, and cyclic alkenyl groups, and further wherein said alkenyl groups are optionally substituted; and
n is 100 or greater; and
(ii) at least one salt of a fatty acid gelling agent;
wherein said at least one PEG fatty acid diester and said at least one salt of a fatty acid gelling agent are present in an amount effective to provide a stable composition.

2. The composition according to claim 1, wherein R is chosen from alkyl groups comprising from 19 to 31 carbon atoms and alkenyl groups comprising from 19 to 31 carbon atoms.

3. The composition according to claim 2, wherein R is chosen from alkyl groups comprising 19 carbon atoms and alkenyl groups comprising 19 carbon atoms.

4. The composition according to claim 2, wherein R is chosen from alkyl groups comprising 20 carbon atoms and alkenyl groups comprising 20 carbon atoms.

5. The composition according to claim 2, wherein R is chosen from alkyl groups comprising 21 carbon atoms and alkenyl groups comprising 21 carbon atoms.

6. The composition according to claim 2, wherein R is chosen from alkyl groups comprising 22 carbon atoms and alkenyl groups comprising 22 carbon atoms.

7. The composition according to claim 2, wherein R is chosen from alkyl groups comprising 23 carbon atoms and alkenyl groups comprising 23 carbon atoms.

8. The composition according to claim 1, wherein n ranges from 100 to 500.

9. The composition according to claim 8, wherein n is 100.

10. The composition according to claim 8, wherein n is 150.

11. The composition according to claim 8, wherein n is 200.

12. The composition according to claim 8, wherein n is 250.

13. The composition according to claim 8, wherein n is 300.

14. The composition according to claim 1, wherein R is chosen from alkyl groups comprising 21 carbon atoms and alkenyl groups comprising 21 carbon atoms, and n is 100.

15. The composition according to claim 1, wherein R is chosen from alkyl groups comprising 21 carbon atoms and alkenyl groups comprising 21 carbon atoms, and n is 150.

16. The composition according to claim 1, wherein said at least one PEG fatty acid diester is present in an amount ranging from 0.001% to 2% by weight relative to the total weight of the composition.

17. The composition according to claim 1, wherein said at least one PEG fatty acid diester is present in an amount ranging up to 15% by weight relative to the total weight of the composition.

18. The composition according to claim 1, wherein said at least one PEG fatty acid diester has a melting point ranging from 50° C. to 60° C.

19. The composition according to claim 18, wherein said at least one PEG fatty acid diester has a melting point ranging from 52° C. to 57° C.

20. The composition according to claim 1, wherein said composition comprises at least two PEG fatty acid diesters of formula (1), wherein R is chosen from alkyl groups comprising at least 19 carbon atoms and alkenyl groups comprising at least 19 carbon atoms, and n is 100 or greater.

21. The composition according to claim 1, wherein said at least one salt of a fatty acid gelling agent is chosen from salts of C₁₀ to C₄₀ fatty acid gelling agents.

22. The composition according to claim 21, wherein said at least one salt of a fatty acid gelling agent is chosen from salts of C₁₂ to C₂₂ fatty acid gelling agents.

23. The composition according to claim 22, wherein said at least one salt of a fatty acid gelling agent is chosen from salts of C₁₄ to C₂₀ fatty acid gelling agents.

24. The composition according to claim 23, wherein said at least one salt of a fatty acid gelling agent is chosen from salts of C₁₆ to C₂₀ fatty acid gelling agents.

25. The composition according to claim 21, wherein said at least one salt of a fatty acid gelling agent is chosen from salts of lauric acid, salts of palmitic acid, salts of stearic acid, and salts of behenic acid.

26. The composition according to claim 1, wherein said at least one salt of a fatty acid gelling agent is chosen from inorganic salts of a fatty acid gelling agent and organic salts of a fatty acid gelling agent.

27. The composition according to claim 26, wherein said inorganic salts of a fatty acid gelling agent are chosen from alkali metal salts of a fatty acid gelling agent.

28. The composition according to claim 27, wherein said alkali metal salts of a fatty acid gelling agent are chosen from sodium salts of a fatty acid gelling agent and potassium salts of a fatty acid gelling agent.

29. The composition according to claim 1, wherein said at least one salt of a fatty acid gelling agent are chosen from sodium laurate and potassium laurate.

30. The composition according to claim 1, wherein said at least one salt of a fatty acid gelling agent are chosen from sodium stearate and potassium stearate.

31. The composition according to claim 1, wherein said at least one salt of a fatty acid gelling agent are chosen from sodium behenate and potassium behenate.

32. The composition according to claim 1, wherein said composition comprises at least two salts of a fatty acid gelling agent.

33. The composition according to claim 32, wherein said at least two salts of a fatty acid gelling agent are chosen from salts of stearic acid and salts of lauric acid.

34. The composition according to claim 1, wherein said at least one salt of a fatty acid gelling agent is present in an amount ranging from 1% to 30% by weight relative to the total weight of the composition.

35. The composition according to claim 34, wherein said at least one salt of a fatty acid gelling agent is present in an amount ranging from 3% to 15% by weight relative to the total weight of the composition.

36. The composition according to claim 1, further comprising water.

37. The composition according to claim 36, wherein said water is present in said composition in an amount ranging from at least 5% by weight relative to the total weight of the composition.

38. The composition according to claim 37, wherein said water is present in said composition in an amount ranging from at least 30% by weight relative to the total weight of the composition.

39. The composition according to claim 1, further comprising at least one PEG fatty acid diester of formula (1), wherein R is chosen from (a) alkyl groups comprising less than 19 carbon atoms, wherein said alkyl groups are chosen from linear alkyl groups, branched alkyl groups, and cyclic alkyl groups, and further wherein said alkyl groups are optionally substituted, and (b) alkenyl groups comprising less than 19 carbon atoms, wherein said alkenyl groups are chosen from linear alkenyl groups, branched alkenyl groups, and cyclic alkenyl groups, and further wherein said alkenyl groups are optionally substituted.

40. The composition according to claim 1, further comprising at least one PEG fatty acid diester of formula (1) wherein R is chosen from (a) alkyl groups comprising from 13 to 18 carbon atoms, wherein said alkyl groups are chosen from linear alkyl groups, branched alkyl groups, and cyclic alkyl groups, and further wherein said alkyl groups are optionally substituted, and (b) alkenyl groups comprising from 13 to 18 carbon atoms, wherein said alkenyl groups are chosen from linear alkenyl groups, branched alkenyl groups, and cyclic alkenyl groups, and further wherein said alkenyl groups are optionally substituted.

41. The composition according to claim 1, further comprising at least one PEG fatty acid diester of formula (1) wherein R is chosen from (a) alkyl groups comprising 17 carbon atoms, wherein said alkyl groups are chosen from linear alkyl groups, branched alkyl groups, and cyclic alkyl groups, and further wherein said alkyl groups are optionally substituted, and (b) alkenyl groups comprising 17 carbon atoms, wherein said alkenyl groups are chosen from linear alkenyl groups, branched alkenyl groups, and cyclic alkenyl groups, and further wherein said alkenyl groups are optionally substituted.

42. The composition according to claim 1, further comprising at least one PEG fatty acid diester of formula (1) wherein R is chosen from (a) alkyl groups comprising 18 carbon atoms, wherein said alkyl groups are chosen from linear alkyl groups, branched alkyl groups, and cyclic alkyl groups, and further wherein said alkyl groups are optionally substituted, and (b) alkenyl groups comprising 18 carbon atoms, wherein said alkenyl groups are chosen from linear alkenyl groups, branched alkenyl groups, and cyclic alkenyl groups, and further wherein said alkenyl groups are optionally substituted.

43. The composition according to claim 1, wherein said at least one PEG fatty acid diester and said at least one salt of a fatty acid gelling agent are present in a system chosen from an aqueous system, an alcohol aqueous system, a hydro-alcoholic system, an oil-in-water emulsion, a water-in-oil, an oil-in-water-in-oil emulsion, and a water-in-oil-in-water emulsion.

44. The composition according to claim 1, wherein said composition is in a form chosen from molded sticks, poured sticks and gels.

45. The composition according to claim 1, wherein said at least one PEG fatty acid diester and said at least one salt of a fatty acid gelling agent are present in an amount effective to further provide hardness to the composition.

46. The composition according to claim 44, wherein said hardness ranges from 10 g to 5000 g.

47. The composition according to claim 45, wherein said hardness ranges from 15 g to 500 g.

48. The composition according to claim 44, wherein said hardness ranges from 20 g to 600 g.

49. The composition according to claim 47, wherein said hardness ranges from 30 g to 150 g.

50. The composition according to claim 1, wherein said composition has a melting point ranging from 40° C. to 150° C.

51. The composition according to claim 1, further comprising at least one additive.

52. The composition according to claim 50, wherein said at least one additive is chosen from fatty materials, waxes, coloring agents, fillers, humectants, texture modifiers, moisturizers, viscosity modifiers, antioxidants, essential oils, preserving agents, fragrances, neutralizing agents, liposoluble polymers, polysaccharides, silicones, fluorinated compounds, and cosmetically active agents and dermatological active agents.

53. The composition according to claim 52, wherein said active agents are chosen from emollients, moisturizers, vitamins, essential fatty acids and UV-screening agents.

54. The composition according to claim 1, further comprising at least one HENDERSON additive with the proviso that said at least one additive is not chosen from polysaccharides.

55. The composition according to claim 1, further comprising at least one additive chosen from glycerin, glycols, and monosaccharides.

56. The composition according to claim 55, wherein said glycols are chosen from propylene glycol and butylene glycol.

57. The composition according to claim 52, wherein said coloring agents are chosen from pigments, dyes, nacreous pigments, and pearling agents.

58. The composition according to claim 52, wherein said pigments are chosen from white inorganic pigments, white organic pigments, colored inorganic pigments, and colored organic pigments, wherein said pigments may be coated or uncoated, and further wherein said pigments may be polymeric or nonpolymeric.

59. The composition according to claim 52, wherein said nacreous pigments are chosen from white nacreous pigments and colored nacreous pigments.

60. The composition according to claim 52, wherein said waxes are chosen from waxes of natural origin, hydrogenated oils, jojoba esters, waxes of synthetic origin, waxes obtained by Fischer-Tropsch synthesis, fatty acid esters other than the at least one PEG fatty acid diester of formula (1), fatty acid glycerides, and silicone waxes.

61. The composition according to claim 1, wherein said composition is not a deodorant product.

62. The composition according to claim 1, wherein said composition is transparent.

63. The composition according to claim 1, wherein said composition is clear.

64. The composition according to claim 1, wherein said composition is not transparent, and further wherein said composition is not clear.

65. The composition according to claim 1, wherein said composition is a foundation, a lipstick, a blusher, a make-up-removing product, a make-up product for the body or the hair, an eyeshadow, an eyeliner, a concealer product, a deodorant product, an antisun product or a care product for the skin, lips or hair.

66. A composition comprising
(i) at least one PEG fatty acid diester of formula (1): 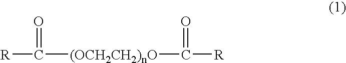
wherein:
R is chosen from (a) alkyl groups comprising at least 19 carbon atoms, wherein said alkyl groups are chosen from linear alkyl groups, branched alkyl groups, and cyclic alkyl groups, and further wherein said alkyl groups are optionally substituted, and (b) alkenyl groups comprising at least 19 carbon atoms, wherein said alkenyl groups are chosen from linear alkenyl groups, branched alkenyl groups, and cyclic alkenyl groups, and further wherein said alkenyl groups are optionally substituted; and
n is 100 or greater;
(ii) at least one salt of a fatty acid gelling agent; and
(iii) water,
wherein said at least one PEG fatty acid diester and said at least one salt of a fatty acid gelling agent are present in an amount effective to provide a solid, stable composition, and
further wherein said water is present in an amount greater than 50% by weight relative to the total weight of the composition.

67. A method for caring for, making up and/or treating at least one keratinous material comprising applying to said at least one keratinous material a cosmetic composition comprising:
(i) at least one PEG fatty acid diester of formula (1): 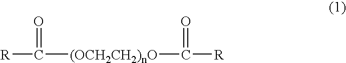
wherein:
R is chosen from (a) alkyl groups comprising at least 19 carbon atoms, wherein said alkyl groups are chosen from linear alkyl groups, branched alkyl groups, and cyclic alkyl groups, and further wherein said alkyl groups are optionally substituted, and (b) alkenyl groups comprising at least 19 carbon atoms, wherein said alkenyl groups are chosen from linear, branched alkenyl groups, and cyclic alkenyl groups, and further wherein said alkenyl groups are optionally substituted; and
n is 100 or greater; and
(ii) at least one salt of a fatty acid gelling agent;
wherein said at least one PEG fatty acid diester and said at least one salt of a fatty acid gelling agent are present in an amount effective to provide a stable composition.

68. A method for modifying the structure of a cosmetic composition which comprises at least one salt of a fatty acid gelling agent, comprising including in said cosmetic composition at least one PEG fatty acid diester of formula (1): 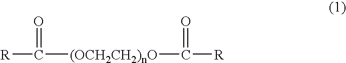
wherein:
R is chosen from (a) alkyl groups comprising at least 19 carbon atoms, wherein said alkyl groups are chosen from linear alkyl groups, branched alkyl groups, and cyclic alkyl groups, and further wherein said alkyl groups are optionally substituted, and (b) alkenyl groups comprising at least 19 carbon atoms, wherein said alkenyl groups are chosen from linear alkenyl groups, branched alkenyl groups, and cyclic alkenyl groups, and further wherein said alkenyl groups are optionally substituted; and
n is 100 or greater, in an amount effective to provide a stable composition.

69. A method for providing stability to a cosmetic composition comprising including in said cosmetic composition:
(i) at least one PEG fatty acid diester of formula (1): 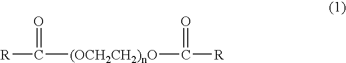
wherein:
R is chosen from (a) alkyl groups comprising at least 19 carbon atoms, wherein said alkyl groups are chosen from linear alkyl groups, branched alkyl groups, and cyclic alkyl groups, and further wherein said alkyl groups are optionally substituted, and (b) alkenyl groups comprising at least 19 carbon atoms, wherein said alkenyl groups are chosen from linear alkenyl groups, branched alkenyl groups, and cyclic alkenyl groups, and further wherein said alkenyl groups are optionally substituted; and
n is 100 or greater; and
(ii) at least one salt of a fatty acid gelling agent;
wherein said at least one PEG fatty acid diester and said at least one salt of a fatty acid gelling agent are present in an amount effective to provide a stable composition.

70. A method for making a cosmetic composition comprising adding to said cosmetic composition:
(i) at least one PEG fatty acid diester of formula (1): 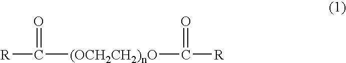
wherein:
R is chosen from (a) alkyl groups comprising at least 19 carbon atoms, wherein said alkyl groups are chosen from linear alkyl groups, branched alkyl groups, and cyclic alkyl groups, and further wherein said alkyl groups are optionally substituted, and (b) alkenyl groups comprising at least 19 carbon atoms, wherein said alkenyl groups are chosen from linear alkenyl groups, branched alkenyl groups, and cyclic alkenyl groups, and further wherein said alkenyl groups are optionally substituted; and
n is 100 or greater; and
(ii) at least one salt of a fatty acid gelling agent;
wherein said at least one PEG fatty acid diester and said at least one salt of a fatty acid gelling agent are present in an amount effective to provide a stable composition.

71. A composition comprising
(i) at least one PEG fatty acid diester of formula (1): 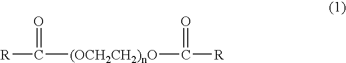
wherein:
R is chosen from (a) alkyl groups comprising at least 19 carbon atoms, wherein said alkyl groups are chosen from linear alkyl groups, branched alkyl groups, and cyclic alkyl groups, and further wherein said alkyl groups are optionally substituted, and (b) alkenyl groups comprising at least 19 carbon atoms, wherein said alkenyl groups are chosen from linear alkenyl groups, branched alkenyl groups, and cyclic alkenyl groups, and further wherein said alkenyl groups are optionally substituted; and
n is 100 or greater; and
(ii) at least one dissociated salt of a fatty acid gelling agent,;
wherein said at least one PEG fatty acid diester and said at least one dissociated salt of a fatty acid gelling agent are present in an amount effective to provide a stable composition.

72. The composition according to claim 71, wherein R is chosen from alkyl groups comprising from 19 to 31 carbon atoms and alkenyl groups comprising from 19 to 31 carbon atoms.

73. The composition according to claim 72, wherein R is chosen from alkyl groups comprising 19 carbon atoms and alkenyl groups comprising 19 carbon atoms.

74. The composition according to claim 72, wherein R is chosen from alkyl groups comprising 20 carbon atoms and alkenyl groups comprising 20 carbon atoms.

75. The composition according to claim 72, wherein R is chosen from alkyl groups comprising 21 carbon atoms and alkenyl groups comprising 21 carbon atoms.

76. The composition according to claim 72, wherein R is chosen from alkyl groups comprising 22 carbon atoms and alkenyl groups comprising 22 carbon atoms.

77. The composition according to claim 72, wherein R is chosen from alkyl groups comprising 23 carbon atoms and alkenyl groups comprising 23 carbon atoms.

78. The composition according to claim 71, wherein n ranges from 100 to 500.

79. The composition according to claim 78, wherein n is 100.

80. The composition according to claim 78, wherein n is 150.

81. The composition according to claim 78, wherein n is 200.

82. The composition according to claim 78, wherein n is 250.

83. The composition according to claim 78, wherein n is 300.

84. The composition according to claim 71, wherein R is chosen from alkyl groups comprising 21 carbon atoms and alkenyl groups comprising 21 carbon atoms, and n is 100.

85. The composition according to claim 71, wherein R is chosen from alkyl groups comprising 21 carbon atoms and alkenyl groups comprising 21 carbon atoms, and n is 150.

86. The composition according to claim 71, wherein said at least one PEG fatty acid diester is present in an amount ranging from 0.001% to 2% by weight relative to the total weight of the composition.

87. The composition according to claim 71, wherein said at least one PEG fatty acid diester is present in an amount ranging up to 15% by weight relative to the total weight of the composition.

88. The composition according to claim 71, wherein said at least one PEG fatty acid diester has a melting point ranging from 50° C. to 60° C.

89. The composition according to claim 88, wherein said at least one PEG fatty acid diester has a melting point ranging from 52° C. to 57° C.

90. The composition according to claim 71, wherein said composition comprises at least two PEG fatty acid diesters of formula (1), wherein R is chosen from alkyl groups comprising at least 19 carbon atoms and alkenyl groups comprising at least 19 carbon atoms, and n is 100 or greater.

91. The composition according to claim 71, wherein said at least one salt of a fatty acid gelling agent is chosen from salts of C₁₀ to C₄₀ fatty acid gelling agents.

92. The composition according to claim 91, wherein said at least one salt of a fatty acid gelling agent is chosen from salts of C₁₂ to C₂₂ fatty acid gelling agents.

93. The composition according to claim 92, wherein said at least one salt of a fatty acid gelling agent is chosen from salts of C₁₄ to C₂₀ fatty acid gelling agents.

94. The composition according to claim 93, wherein said at least one salt of a fatty acid gelling agent is chosen from salts of C₁₆ to C₂₀ fatty acid gelling agents.

95. The composition according to claim 92, wherein said at least one salt of a fatty acid gelling agent is chosen from salts of lauric acid, salts of palmitic acid, salts of stearic acid, and salts of behenic acid.

96. The composition according to claim 71, wherein said at least one salt of a fatty acid gelling agent is chosen from inorganic salts of a fatty acid gelling agent and organic salts of a fatty acid gelling agent.

97. The composition according to claim 96, wherein said inorganic salts of a fatty acid gelling agent are chosen from alkali metal salts of a fatty acid gelling agent.

98. The composition according to claim 97, wherein said alkali metal salts of a fatty acid gelling agent are chosen from sodium salts of a fatty acid gelling agent and potassium salts of a fatty acid gelling agent.

99. The composition according to claim 71, wherein said at least one salt of a fatty acid gelling agent are chosen from sodium laurate and potassium laurate.

100. The composition according to claim 71, wherein said at least one salt of a fatty acid gelling agent are chosen from sodium stearate and potassium stearate.

101. The composition according to claim 71, wherein said at least one salt of a fatty acid gelling agent are chosen from sodium behenate and potassium behenate.

102. The composition according to claim 71, wherein said composition comprises at least two salts of a fatty acid gelling agent.

103. The composition according to claim 102, wherein said at least two salts of a fatty acid gelling agent are chosen from salts of stearic acid and salts of lauric acid.

104. The composition according to claim 71, wherein said at least one salt of a fatty acid gelling agent is present in an amount ranging from 1% to 30% by weight relative to the total weight of the composition.

105. The composition according to claim 104, wherein said at least one salt of a fatty acid gelling agent is present in an amount ranging from 3% to 15% by weight relative to the total weight of the composition.

106. The composition according to claim 71, further comprising water.

107. The composition according to claim 107, wherein said water is present in said composition in an amount ranging from 5% by weight relative to the total weight of the composition.

108. The composition according to claim 71, further comprising at least one PEG fatty acid diester of formula (1) wherein R is chosen from alkyl groups comprising less than 19 carbon atoms and alkenyl groups comprising less than 19 carbon atoms.

109. The composition according to claim 71, further comprising at least one PEG fatty acid diester of formula (1) wherein R is chosen from alkyl groups comprising from 13 to 18 carbon atoms and alkenyl groups comprising from 13 to 18 carbon atoms.

110. The composition according to claim 71, further comprising at least one PEG fatty acid diester of formula (1) wherein R is chosen from alkyl groups comprising 17 carbon atoms and alkenyl groups comprising 17 carbon atoms.

111. The composition according to claim 71, further comprising at least one PEG fatty acid diester of formula (1) wherein R is chosen from alkyl groups comprising 18 carbon atoms and alkenyl groups comprising 18 carbon atoms.

112. A composition formed from:
(i) at least one PEG fatty acid diester of formula (1): 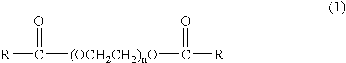
wherein:
R is chosen from (a) alkyl groups comprising at least 19 carbon atoms, wherein said alkyl groups are chosen from linear alkyl groups, branched alkyl groups, and cyclic alkyl groups, and further wherein said alkyl groups are optionally substituted, and (b) alkenyl groups comprising at least 19 carbon atoms, wherein said alkenyl groups are chosen from linear alkyl groups, branched alkyl groups, and cyclic alkyl groups, and further wherein said alkenyl groups are optionally substituted; and
n is 100 or greater; and
(ii) at least one salt of a fatty acid gelling agent;
wherein said at least one PEG fatty acid diester and said at least one salt of a fatty acid gelling agent are present in an amount effective to provide a stable composition.

113. The composition according to claim 112, wherein R is chosen from alkyl groups comprising from 19 to 31 carbon atoms and alkenyl groups comprising from 19 to 31 carbon atoms.

114. The composition according to claim 113, wherein R is chosen from alkyl groups comprising 19 carbon atoms and alkenyl groups comprising 19 carbon atoms.

115. The composition according to claim 113, wherein R is chosen from alkyl groups comprising 20 carbon atoms and alkenyl groups comprising 20 carbon atoms.

116. The composition according to claim 113, wherein R is chosen from alkyl groups comprising 21 carbon atoms and alkenyl groups comprising 21 carbon atoms.

117. The composition according to claim 113, wherein R is chosen from alkyl groups comprising 22 carbon atoms and alkenyl groups comprising 22 carbon atoms.

118. The composition according to claim 113, wherein R is chosen from alkyl groups comprising 23 carbon atoms and alkenyl groups comprising 23 carbon atoms.

119. The composition according to claim 112, wherein n ranges from 100 to 500.

120. The composition according to claim 119, wherein n is 100.

121. The composition according to claim 119, wherein n is 150.

122. The composition according to claim 119, wherein n is 200.

123. The composition according to claim 119, wherein n is 250.

124. The composition according to claim 119, wherein n is 300.

125. The composition according to claim 112, wherein R is chosen from alkyl groups comprising 21 carbon atoms and alkenyl groups comprising 21 carbon atoms, and n is 100.

126. The composition according to claim 112, wherein R is chosen from alkyl groups comprising 21 carbon atoms and alkenyl groups comprising 21 carbon atoms, and n is 150.

127. The composition according to claim 112, wherein said at least one PEG fatty acid diester is present in an amount ranging from 0.001% to 2% by weight relative to the total weight of the composition.

128. The composition according to claim 112, wherein said at least one PEG fatty acid diester is present in an amount ranging up to 15% by weight relative to the total weight of the composition.

129. The composition according to claim 112, wherein said at least one PEG fatty acid diester has a melting point ranging from 50° C. to 60° C.

130. The composition according to claim 129, wherein said at least one PEG fatty acid diester has a melting point ranging from 52° C. to 57° C.

131. The composition according to claim 112, wherein said composition comprises at least two PEG fatty acid diesters of formula (1), wherein R is chosen from alkyl groups comprising at least 19 carbon atoms and alkenyl groups comprising at least 19 carbon atoms, and n is 100 or greater.

132. The composition according to claim 112, wherein said at least one salt of a fatty acid gelling agent is chosen from salts of C₁₀ to C₄₀ fatty acid gelling agents.

133. The composition according to claim 132, wherein said at least one salt of a fatty acid gelling agent is chosen from salts of C₁₂ to C₂₂ fatty acid gelling agents.

134. The composition according to claim 133, wherein said at least one salt of a fatty acid gelling agent is chosen from salts of C₁₄ to C₂₀ fatty acid gelling agents.

135. The composition according to claim 134, wherein said at least one salt of a fatty acid gelling agent is chosen from salts of C₁₆ to C₂₀ fatty acid gelling agents.

136. The composition according to claim 132, wherein said at least one salt of a fatty acid gelling agent is chosen from salts of lauric acid, salts of palmitic acid, salts of stearic acid, and salts of behenic acid.

137. The composition according to claim 112, wherein said at least one salt of a fatty acid gelling agent is chosen from inorganic salts of a fatty acid gelling agent and organic salts of a fatty acid gelling agent.

138. The composition according to claim 137, wherein said inorganic salts of a fatty acid gelling agent are chosen from alkali metal salts of a fatty acid gelling agent.

139. The composition according to claim 138, wherein said alkali metal salts of a fatty acid gelling agent are chosen from sodium salts of a fatty acid gelling agent and potassium salts of a fatty acid gelling agent.

140. The composition according to claim 112, wherein said at least one salt of a fatty acid gelling agent are chosen from sodium laurate and potassium laurate.

141. The composition according to claim 112, wherein said at least one salt of a fatty acid gelling agent are chosen from sodium stearate and potassium stearate.

142. The composition according to claim 112, wherein said at least one salt of a fatty acid gelling agent are chosen from sodium behenate and potassium behenate.

143. The composition according to claim 112, wherein said composition comprises at least two salts of a fatty acid gelling agent.

144. The composition according to claim 143, wherein said at least two salts of a fatty acid gelling agent are chosen from salts of stearic acid and salts of lauric acid.

145. The composition according to claim 112, wherein said at least one salt of a fatty acid gelling agent is present in an amount ranging from 1% to 30% by weight relative to the total weight of the composition.

146. The composition according to claim 145, wherein said at least one salt of a fatty acid gelling agent is present in an amount ranging from 3% to 15% by weight relative to the total weight of the composition.

147. The composition according to claim 112, further comprising water.

148. The composition according to claim 147, wherein said water is present in said composition in an amount ranging from 5% by weight relative to the total weight of the composition.

149. The composition according to claim 112, further comprising at least one PEG fatty acid diester of formula (1) wherein R is chosen from alkyl groups comprising less than 19 carbon atoms and alkenyl groups comprising less than 19 carbon atoms.

150. The composition according to claim 112, further comprising at least one PEG fatty acid diester of formula (1) wherein R is chosen from alkyl groups comprising from 13 to 18 carbon atoms and alkenyl groups comprising from 13 to 18 carbon atoms.

151. The composition according to claim 112, further comprising at least one PEG fatty acid diester of formula (1) wherein R is chosen from alkyl groups comprising 17 carbon atoms and alkenyl groups comprising 17 carbon atoms.

152. The composition according to claim 112, further comprising at least one PEG fatty acid diester of formula (1) wherein R is chosen from alkyl groups comprising 18 carbon atoms and alkenyl groups comprising 18 carbon atoms.
